(19) United States
(12) Patent Application Publication
(10) Pub. No.: US 2019/0380659 A1
(43) Pub. Date: Dec. 19, 2019

(54) FUTURE-IMAGE PREDICTION DEVICE

(71) Applicants: Rika OHKUMA, Palo Alto, CA (US); Akiko SVENSSON KISHI, Tokyo (JP)

(72) Inventors: Rika OHKUMA, Palo Alto, CA (US); Akiko SVENSSON KISHI, Tokyo (JP)

(21) Appl. No.: 16/097,878

(22) PCT Filed: May 8, 2017
(86) PCT No.: PCT/JP2017/017350

§ 371 Date: Feb. 4, 2019

(30) Foreign Application Priority Data
May 4, 2016 (JP) 2016-092870

(51) Int. Cl. A61B 5/00; G06K 9/00; A61B 5/1171

(52) U.S. Cl. CPC A61B 5/7275; G06K 9/00281; A61B 5/1176; G06K 9/00255; G06K 9/00275

(57) ABSTRACT

[Object]

To provide a future-image predicting device for predicting a future-image of a target person caused by a predetermined disease

[Solution]

A future-image predicting device 1 includes a storing unit 3, an input unit 2, and a control unit 4. The storing unit 3 stores a first database which indicates a relation between health-related data and a risk level of Metabolic syndrome, and a second data base which indicates a relation between the risk level of Metabolic syndrome and a change tendency in an appearance or an organ. Health-related data and appearance data or organ data of a target person are entered into the input unit 2. The control unit 4 determines a risk level of Metabolic syndrome for the health-related data of the target person, while referring to the first database; determines a change tendency in an appearance or an organ of the target person, based on the determined risk level, while referring to the second database; and generates a future-image of the target person in an appearance or an organ, based on the appearance data or organ data and the determined change tendency.

### TECHNICAL FIELD OF THE INVENTION

The present invention relates to a future-image predicting device for predicting a future-image of a target person caused by a predetermined disease.

Conventionally, an information terminal device, which generates health management data with respect to change in skin color by comparing the user's current face-image with the user's past face-image, is known (PTL1).

### PRIOR ART

PTL1: Japanese Unexamined Patent Application No. 2014-180350

### SUMMARY OF INVENTION

### Problem to be Solved by the Invention

However, the above device does not predict the risk of any disease concretely, while judging the health condition of the user in consideration of the health management data generated only based on the change in the face-image. Therefore, it is less likely that the above device motivates the user to improve awareness and behavior for health management. Further, the above device, which judges only the current health condition, does not predict the possibility of disease.

In view of the foregoing, it is an object of the invention to provide a future-image predicting device capable of strongly motivating a target person to improve awareness and behavior for health management.

### Means for Solving the Problem

In order to solve the problem, the present invention provides a future-image predicting device including: a storing unit configured to store a first database which indicates a relation between health-related data and a risk level of a predetermined disease, and a second data base which indicates a relation between the risk level and a change tendency in an appearance or an organ; an input unit into which health-related data and appearance data or organ data of a target person are entered; a first determining unit configured to determine a risk level of the predetermined disease for the health-related data of the target person, while referring to the first database; a second determining unit configured to determine a change tendency in an appearance or an organ of the target person, based on the determined risk level, while referring to the second database; and an image generating unit configured to generate a future-image of the target person in an appearance or an organ, based on the appearance data or organ data and the determined change tendency.

With this construction, the target person can know visually the future-image of one's own if the current health management is continued. As the result, the target person can be strongly motivated to improve awareness and behavior for health management.

It is preferable that the storing unit stores the risk level and the change tendency for each type of the health-related data. The first determining unit determines the risk level for each type of the health-related data. The second determining unit determines the change tendency for each type of the health-related data. The image generating unit generates the future-image, based on the appearance data or organ data of the target person and the change tendency determined for each type of the health-related data.

With this construction, the health-related data, which is closely relevant to the predetermined disease, has greater influence on the determination of the risk level of the predetermined disease and the change tendency. Consequently, the determination of the risk level of the predetermined disease and the change tendency in appearance or organ are expected to be highly reliable.

It is preferable that future-image predicting device further includes a first display unit configured to display the future-image. The display unit is capable of further displaying at least one of a first operation part capable of continuously or stepwisely changing the health-related data of the target person and a second operation part capable of continuously or stepwisely changing the future-image. When the health-related data is changed with the first operation part, a future-image corresponding to the changed health-related data is displayed on the first display unit. When the future-image is changed with the second operation part, health-related data corresponding to the changed future-image is displayed on the first display unit.

With this construction, the change in the appearance or in the organ of the target person, which is expected through improvement of the awareness and the behavior for health management, is indicated visually and intuitively. Thus, the target person is given a more strong motivation to improve awareness and the behavior for health management.

It is preferable that future-image predicting device further includes an output unit configured to output the future-image. The first determining unit calculates a composite value of a plurality of risk levels respectively determined for a plurality of types of the health-related data. The storing unit further stores a third database which indicates a relation between the composite value and a developing-risk or a severed-risk of the predetermined disease. The output unit outputs the developing-risk or the severed-risk of the predetermined disease corresponding to the composite value the plurality of risk levels obtained from the health-related data of the target person.

With this construction, the “developing-risk or severed-risk” caused if the current health management is continued is shown with numerals or letters. As the result, the target person is more strongly motivated to improve the awareness and the behavior for health management.

It is preferable that future-image predicting device further includes a second display unit configured to selectably display a plurality of organ images. An organ image selected on the second display unit is entered into the input unit as the organ data of the target person.

With this construction, it becomes possible to generate the future-image even if it is not easy to acquire the image data.

Another aspect of the present invention provides a program instructions installed on and executed by a computer for controlling a computer including: a storing unit configured to store a first database which indicates a relation between health-related data and a risk level of a predetermined disease, and a second data base which indicates a relation between the risk level and a change tendency in an appearance or an organ; and an input unit into which health-related data and appearance data or organ data of a target person are entered. The program includes: a first step for determining a risk level of the predetermined disease for the health-related data of the target person, while referring to the first database; a second step for determining a change tendency in an appearance or an organ of the target person, based on the determined risk level, while referring to the second database; and a third step for generating a future-image of the target person in an appearance or an organ, based on both the appearance data or organ data and the determined change tendency.

It is preferable that the storing unit stores the risk level and the change tendency for each type of the health-related data. In the first step, the first determining unit determines the risk level for each type of the health-related data. In the second step, the second determining unit determines the change tendency for each type of the health-related data. In the third step, the image generating unit generates the future-image, based on the appearance data or organ data of the target person and the change tendency determined for each type of the health-related data.

It is preferable that the computer further includes a first display unit configured to display the future-image. The display unit is capable of further displaying at least one of a first operation part capable of continuously or stepwisely changing the health-related data of the target person and a second operation part capable of continuously or stepwisely changing the future-image. The program further includes a fourth step for displaying, when the health-related data is changed with the first operation part, a future-image corresponding to the changed health-related data on the first display unit, and for displaying, when the future-image is changed with the second operation part, health-related data corresponding to the changed future-image on the first display unit.

It is preferable that the computer further includes an output unit configured to output the future-image. A composite value of a plurality of risk levels respectively determined for a plurality of types of the health-related data is calculated. The storing unit further stores a third database which indicates a relation between a composite value and a developing-risk or a severed-risk of the predetermined disease. The program further includes a fifth step for outputting, from the output unit, the developing-risk or the severed-risk of the predetermined disease corresponding to the calculated composite value.

It is preferable that the computer further includes a second display unit configured to selectably display a plurality of organ images. An organ image selected on the second display unit is entered into the input unit as the organ data of the target person.

Another aspect of the present invention provides a non-transitory computer readable storage medium storing a set of program instructions installed on and executed by a computer for controlling a computer including: a storing unit configured to store a first database which indicates a relation between health-related data and a risk level of a predetermined disease, and a second data base which indicates a relation between the risk level and a change tendency in an appearance or an organ; and an input unit into which health-related data and appearance data or organ data of a target person are entered. The program includes: a first step for determining a risk level of the predetermined disease for the health-related data of the target person, while referring to the first database; a second step for determining a change tendency in an appearance or an organ of the target person, based on the determined risk level, while referring to the second database; and a third step for generating a future-image of the target person in an appearance or an organ, based on both the entered appearance data or the organ data and the determined change tendency.

It is preferable that the storing unit stores the risk level and the change tendency for each type of the health-related data. In the first step, the first determining unit determines the risk level for each type of the health-related data. In the second step, the second determining unit determines the change tendency for each type of the health-related data. In the third step, the image generating unit generates the future-image, based on the appearance data or organ data of the target person and the change tendency determined for each type of the health-related data.

It is preferable that the computer further includes a first display unit configured to display the future-image. The display unit is capable of further displaying at least one of a first operation part capable of continuously or stepwisely changing the entered health-related data of the target person and a second operation part capable of continuously or stepwisely changing the future-image. The program further includes a fourth step for displaying, when the health-related data is changed with the first operation part, a future-image corresponding to the changed health-related data on the first display unit, and for displaying, when the future-image is changed with the second operation part, health-related data corresponding to the changed future-image on the first display unit.

It is preferable that the computer further includes an output unit configured to output the future-image. A composite value of a plurality of risk levels respectively determined for a plurality of types of the health-related data is calculated. The storing unit further stores a third database which indicates a relation between a composite value and a developing-risk or a severed-risk of the predetermined disease. The program further includes a fifth step for outputting, from the output unit, the developing-risk or the severed-risk of the predetermined disease corresponding to the calculated composite value.

It is preferable that the computer further includes a second display unit configured to selectably display a plurality of organ images. An organ image selected on the second display unit is entered into the input unit as the organ data of the target person.

### Effects of the Invention

The future-image predicting device of the present invention is capable of strongly motivating a target person to improve awareness and behavior of health management.

### BRIEF DESCRIPTION OF THE DRAWINGS

FIG. 1 is a block diagram showing a configuration of the future-image predicting device, according to a preferred embodiment of the present invention.

FIG. 2 is an explanatory drawing of “relation between health-related data and risk level of Metabolic syndrome”, according to the preferred embodiment of the present invention.

FIG. 3 is an explanatory drawing of “risk level of Metabolic syndrome and change tendency in appearance”.

FIG. 4 shows a future-image according to the preferred embodiment of the present invention.

FIG. 5 is a flowchart with respect to operations of a control unit, according to the preferred embodiment of the present invention.

FIG. 6 is an explanatory drawing of a future-image according to a modification of the present invention.

FIG. 7 is an explanatory drawing of changing of a future-image according to another modification of the present invention.

### PREFERRED EMBODIMENTS

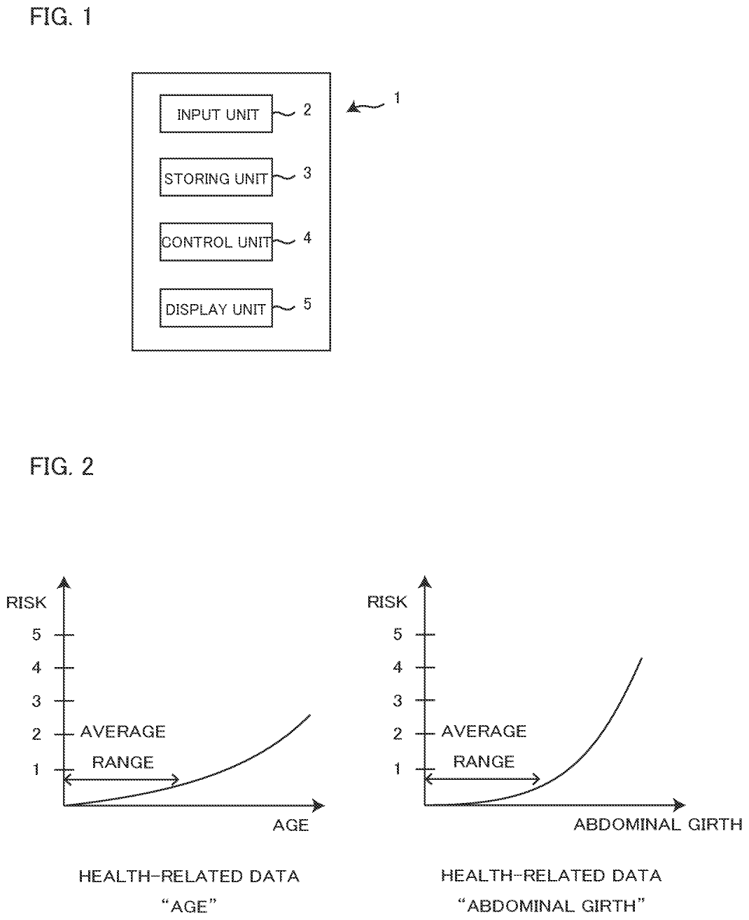

A future-image predicting device 1 according to a preferred embodiment of the present invention will be described below, while referring to the accompanying drawings, FIGS. 1 to 5.

The future-image predicting device 1 determines a risk of a predetermined disease on a target person, based on health-related data of the target person, such as biological data and life-habit data, and predicts a future-image of the target person caused by the predetermined disease. In the present embodiment, as one example, the case where the future-image predicting device 1 predicts the appearance of the target person caused by Metabolic syndrome, is described.

As shown in FIG. 1, the future-image predicting device 1 includes; an input unit 2, a storing unit 3, a control unit 4 and a display unit 5.

Health-related data and appearance data taken from the target person is entered into the input unit 2.

The health-related data can include, for example, basic data of the target person such as “age” and “sex”; biological data such as “blood data”, “blood pressure”, and “abdominal girth”; life-habit data such as “alcohol drinking”, “smoking”, and “exercise”; disease data such as “past medical history”, “disease under medical treatment”, and “disease history of family”. Not only data obtained from medical checkups but any data which can be relevant to diseases may be included in the health-related data. Further, image data of the target person, such as photograph data, is entered as the appearance data, in the present embodiment.

In the storing unit 3, a first database with respect to “relation between health-related data and risk level of Metabolic syndrome” and a second database with respect to “relation between risk level of Metabolic syndrome and change tendency in appearance” are stored. These database are sourced to, for example, medical data accumulated in medical institutions (EMR: Electronic Medical Record), various database accumulated in data correcting companies, national health insurance association or employment-based health insurance association, and personal data which the target person has privately (PHR: Personal Health Record). In the present embodiment, the storing unit 3 is connected to the database of the medical institution which preserves medical checkup results. The storing unit 3 automatically imports the update every time when the database of the medical institution is updated, and automatically updates the first database and the second database. However, the first database and the second database can be manually updated.

The contents of the first database with respect to “relation between health-related data and risk level of Metabolic syndrome” are graphically shown in FIG. 2. As illustrated in FIG. 2, the “relation between health-related data and risk level of Metabolic syndrome” is individually set for each type of the health-related data so that the health-related data, which is closely relevant to the development of Metabolic syndrome, has greater influence on the determination of “risk level of Metabolic syndrome”. For example, in FIG. 2, the “risk level of Metabolic syndrome” for the health-related data “abdominal girth” is set to sharply rise when the “abdominal girth” slightly exceeds the average range, in comparison with the health-related data “age”.

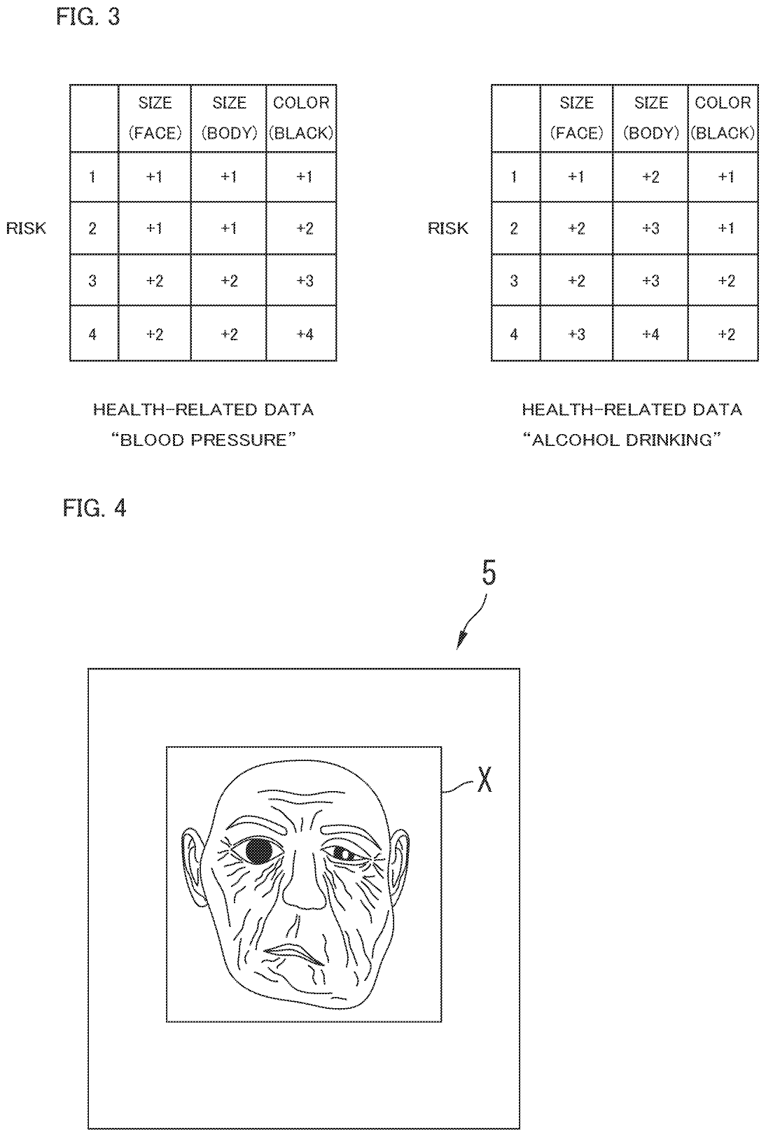

The contents of the second database with respect to “relation between risk level of Metabolic syndrome and change tendency in appearance” are shown in FIG. 3. As elements of the appearance, “size”, “color phase”, “uneven”, “spots”, “wrinkles” etc. can be considered. However, the elements are not limited to the above and any element relevant to the appearance can be employed. The “change tendency in appearance” is individually set for each type of the health-related data so that the health-related data, which is closely relevant to the development of Metabolic syndrome, has greater influence on the determination of “change tendency in appearance”. For example, in FIG. 3, the “change tendency in appearance (size)” for the health-related data “alcohol drinking” is set to be more likely to increase in comparison with the health-related data “blood pressure”. Furthermore, with respect to the health-related data “alcohol drinking”, the “change tendency in appearance (body size)” is set to be more likely to increase than the “change tendency in appearance (face size)”.

A database with respect to “relation between aging and change tendency in appearance” is also stored in the storing unit 3. The “change tendency in appearance” for aging is also individually set for each element in the appearance and is set on each passing year.

The control unit 4 generates a future-image X of the target person caused by Metabolic syndrome, based on the health-related data and the app Inearance data input from the input unit 2, while referring to the first database and the second database stored in the storing unit 3. The future-image X is displayed on the display unit 5, as shown in FIG. 4. Note that the control unit 4 corresponds to the “first determining unit”, the “second determining unit”, and the “image generating unit” of the present invention; and the display unit 5 corresponds to the “first display unit”, the “second display unit”, and the “output unit” of the present invention.

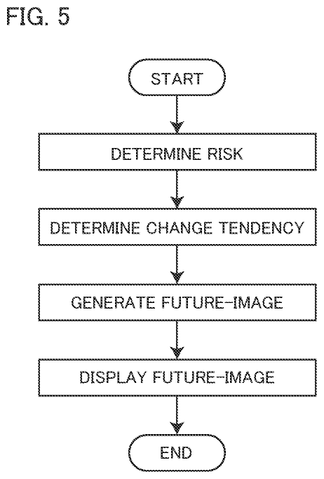

The operation of the control unit 4 will be described in detail, while referring to the flowchart in FIG. 5. The future-image predicting device 1 starts the image generating process, after the health-related data and the appearance data are entered into the input unit 2 and the operator presses down the future-image generating button (not shown) of the future-image predicting device 1.

First, the control unit 4 determines the “risk level of Metabolic syndrome” of the target person based on the health-related data entered into the input unit 2, while referring to the first database stored in the storing unit 3 (S1).

The “risk level of Metabolic syndrome” is determined respectively for each type of the health-related data; such as “risk level (age)”: 1; “risk level (abdominal girth)”: 3, for example.

Next, the control unit 4 determines “change tendency in appearance” based on the “risk level of Metabolic syndrome” determined in S1, while referring to the second database stored in the storing unit 3 (S2).

For example, as shown in FIG. 3, when the “risk level of Metabolic syndrome” for the health-related data “blood pressure” is “1”, the “change tendency” is indicated like, “change tendency (face size)”: “+1”, “change tendency (body size)”: “+1”, and “change tendency (color phase (black))”: “+1”. When the “risk level of Metabolic syndrome” for the health-related data “alcohol drinking” is “2”, the “change tendency” is indicated like, “change tendency (face size)”: “+2”, “change tendency (body size)”: “+3”, and “change tendency (color phase (black))”: “+1”. The control unit 4 determines “change tendency” individually for each type of the health-related data, and calculates a composite value of the plurality of risk levels respectively determined for the plurality of types of the health-related data. In the example of FIG. 3, the composite value of “change tendency” for each element of the appearance is finally obtained like, “change tendency (face size)”: “+3”, “change tendency (body size)”: “+4”, and “change tendency (color phase (black))”: “+2”.

Next, the control unit 4 generates a future-image X, based on both the appearance data entered into the input unit 2 and the “change tendency in appearance” determined in S2 (S3). At this time, it is preferable that the “relation between aging and change tendency in appearance” stored in the storing unit 3 is also taken into account. In this embodiment, the “change tendency in appearance” for aging corresponding to predetermined passing years is automatically added to the “change tendency in appearance” determined in S2. As the result, the future-image X is generated by changing/modifying the image data (appearance data) of the target person based on both the “change tendency in appearance” determined in S2 and the “change tendency in appearance” for aging.

Diabetes, Hypertension, or Hyperlipidemia can be developed due to Metabolic syndrome. When Diabetes, Hypertension, or Hyperlipidemia is severely progressed, they have potential to grow into Obesity, Diabetes-related disease, Cerebral infarction, Cardiac infarction, Cardiac failure, Renal failure, Hepatic failure, Hardening of arteries, Eye disease, Neurological disorder, etc. FIG. 4 shows one example of the future-image X of the target person caused by Metabolic syndrome. In FIG. 4, as the result of Metabolic syndrome progressed, those signs are shown; “accelerated aging” and “weight gain” which can be caused due to metabolic deterioration, “face hemiplegia” which can be followed by Cerebral infarction, “clouded eye lens” due to Cataract which can be followed by severed Diabetes.

In the final step, the generated future-image X is displayed on the display unit 5 (S4).

As described above, the future-image predicting device 1 according to the present embodiment determines the risk of the predetermined disease on the target person, based on the health-related data of the target person such as the biological data and life-habit data. Then, the future-image predicting device 1 generates, based on the determined risk, the future-image X of the target person caused by the predetermined disease. This allows the target person to know visually the future-image of one's own caused if the current health management is continued. As the result, the target person can be strongly motivated to improve awareness and behavior for health management.

Further, in the future-image predicting device 1 of the embodiment, the risk level of the predetermined disease and the change tendency in the appearance are stored for each type of the health-related data. The future-image predicting device 1 determines the risk of the predetermined disease and the change tendency in appearance, individually for each type the health-related data. Such a configuration allows the health-related data, which is closely relevant to the predetermined disease, to have greater influence on the determination of the risk level of the predetermined disease and the change tendency in appearance. Consequently, the determination of the risk level of the predetermined disease and the change tendency in appearance are expected to be highly reliable.

While the future-image predicting device of the invention has been described in detail with reference to the preferred embodiment thereof, it would be apparent to those skilled in the art that many modifications and variations may be made therein without departing from the spirit of the invention, the scope of which is defined by the attached claims.

For example, in the above embodiment, the future-image X of the target person caused by Metabolic syndrome, is generated. However, the future-image X of the target person caused by another disease or a plurality of diseases, can be generated. In this case, when the operator selects a changed part in the future-image X, the name of the disease causing the change may be displayed.

The health-related data is not limited to the foregoing in the embodiment. For example, when the target person develops “Depression”, voice data may be employed as the health-related data. Further, the health-related data measured by a measuring device such as a 3D body scanner or a wearable device can be also employed. As the health-related data measured by the above measuring device, “3D scan data” with respect to body parts such as waist, thigh, or organ parts such as blood vessels; “body composition data” with respect to muscle amount, fat amount, and water content; and “motion tracking data” for the user's movement are considered.

In the above embodiment, the image data such as photos are employed as the appearance data. However, the future-image X generated solely based on the image data is likely to become grotesque and unacceptable to the target person. For this reason, it is also possible to employ pseudo image data such as portraits or avatars instead. Further, two types of future-images X may be generated; one is generated from an image data and the other is from a pseudo data, in order to display them alternately. This configuration makes it possible that the future-image, which is generated solely based on the image data, is displayed, only when it is requested. By employing such a configuration, the future-image X becomes widely accessible to many people.

While the future-image X of the appearance of the target person is generated in the above embodiment, the future-image X of internal organ or blood vessel may be generated. For example, when the target person has “Diabetes”, the future-image X may possibly show such signs as “Nephropathy”, “Retinopathy”, “Periodontal disease”, “Extremities necrosis”, and “Hardening of arteries”.

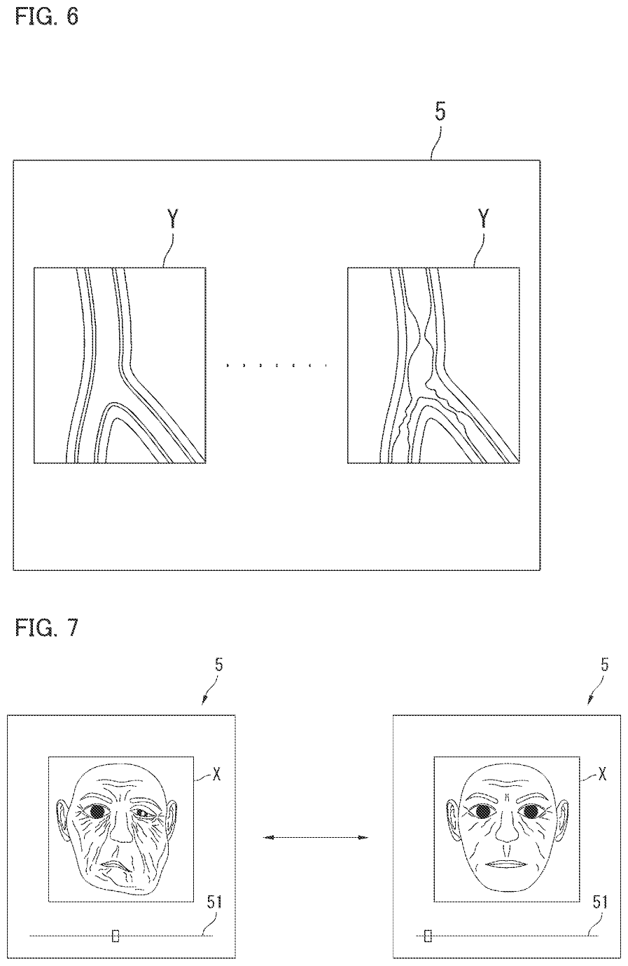

In the case of organ, it is not easy to acquire the image data. Therefore, it is preferable that storing unit 3 stores, in advance, a plurality of blood vessel (organ) images Y, from healthy blood vessel to troubled one, as illustrated in FIG. 6, and the display unit 5 displays the image selectably. When the operator selects one blood vessel (organ) image Y, which is presumed from the health-related data of the target person, the corresponding data of organ can be entered into the input unit 2 as the image data. At that time, the control unit 4 selects one closest blood vessel (organ) image Y, which is predicted from the “risk level of the predetermined disease” and the accompanying “change tendency in appearance” response to the health-related data, among the plurality of blood vessel (organ) image images Y, and displays the selected blood vessel (organ) image Y as the future-image X, on the display unit 5.

The future-image predicting device may be configured to be capable of changing the health-related data after the future-image X is generated. In this case, the display unit 5 displays an operation part 51, as illustrated in FIG. 7. The health-related data of the target person can be changed continuously or stepwisely with the operation part 51. The operation part 51 is considered to be a slide bar and the like. The position of the slide bar corresponds to the value of the health management data. By moving the slide bar, the operator can change the value of health management data, and the future-image X is changed accordingly.

This makes it possible that, when the value of the health management data “smoking” is lowered, the future-image X, which reflects the assumption for the target person to quit a smoking habit, is displayed. In this way, the change in the appearance or in the organ of the target person, which is expected through the improvement of awareness and the behavior for health management, is indicated visually and intuitively. Thus, the target person is given a more strong motivation to improvement the awareness and the behavior for health management.

In reveres, when the future-image X is changed by the operation of the operation part 51, the health-related data corresponding to the changed future-image X may be displayed. Note that the operation part 51 corresponds to the “first operation part” and the “second operation part” of the present invention.

The future-image generating device may determine “developing-risk or severed-risk of the predetermined disease” based on the health-related data of the target person, and display on the display unit 5. In this case, the storing unit 3 stores a third database with respect to a relation between the “composite value of the predetermined disease” and the “developing-risk or severed-risk” of the predetermined disease. After determining the risk level of the predetermined disease for each type of the health-related data, the control unit 4 calculates the composite value of the plurality of risk levels respectively determined for the plurality of types of the health-related data. The output of the “developing-risk or severed-risk” is not only displayed in display unit 5, but may be printed out by a printer.

With this configuration, the “developing-risk or severed-risk” caused if the current health management is continued is shown with numerals or letters. As the result, the target person is more strongly motivated toward the improvement of awareness and the behavior for health management.

In the above embodiment, the future-image X is output by way of displaying it on the display unit 5. However, the future-image X may also be printed out by a printer. The output way of the “output unit” of the present invention further includes projection, space drawing and the like. In this case, any material capable of working as a target of the projection, such as wall, floor, mirror, and space may be employed as the “first display unit” and the “second display unit” of the present invention. Further, the future-image X converted to VR (virtual reality) or AR (augmented reality) may be output. Furthermore, the future-image X may be output with three-dimension or hologram. Monitors, which are provided integrally or separately to an information terminal device or a health-related data measurement device, may also be employed as the “first display unit” and the “second display unit” of the present invention.

In the above embodiment, the display unit 5 plays all these roles of; the “first display unit”, the “second display unit”, and the “output unit”. However, these units may be provided separately.

It is also possible that some parts of the future-image X is selectably displayed, and, when the operator selects the part including some changes in the appearance, a medical advice for the target person to reduce change of the selected part in the future-image X may be displayed.

In the above embodiment, the “risk level of Metabolic syndrome” and the “change tendency in appearance” are stored in the storing unit 3 with predetermined parameters. However, it is also possible that the future-image predicting device 1 is designed to have a learning function for calculating the “risk level of Metabolic syndrome” and the “change tendency in appearance”. In this case, the “risk level of Metabolic syndrome” and the “change tendency in appearance” are stored in the storing unit 3, each time they are calculated.

The present invention is also applied to a program corresponding to the process which the control unit 4 performs, or to a record media storing the content of the program. In the case of the record media, the program should be installed on a computer. As the record media storing the program, a non-transitory computer readable medium may be employed. As the non-transitory readable medium, for example, CD-ROM can be employed, but the record media is not limited to this.

The “future-image predicting device” of the present invention is not limited to a general information terminal device. For example, a portable phone, a smartphone, a portable information terminal, or a measurement device for measuring health-related data can be employed as the “future-image predicting device” of the present invention. Further, a program or an application used in any one of the information terminal device presented above can be employed as the “program” of the present invention.

### DESCRIPTION OF THE REFERENCE NUMBER

1 Future-image predicting device
2 Input unit
3 Storing unit
4 Control unit
5 Display unit
51 Operation unit
X Future-Image

### [Selected Drawing] FIG. 5

### [Translations of Drawings]

### FIG. 1

input unit 2
storing unit 3
control unit 4
display unit 5

### FIG. 2

health-related data “age”: risk (vertical axis), age (horizontal axis), average range (arrow)
health-related data “abdominal girth”: risk (vertical axis), abdominal girth (horizontal axis), average range (arrow)

### FIG. 3

health-related data “blood pressure”: risk, size (face), size (body), color (black)
health-related data “alcohol drinking”: risk, size (face), size (body), color (black)

### FIG. 5

### Start

Determine risk
Determine change tendency
Generate future-image
Display future-image

### End

1. A future-image predicting device comprising:
a storing unit configured to store a first database which indicates a relation between health-related data and a risk level of a predetermined disease, and a second data base which indicates a relation between the risk level and a change tendency in an appearance or an organ;
an input unit into which health-related data and appearance data or organ data of a target person are entered;
a first determining unit configured to determine a risk level of the predetermined disease for the health-related data of the target person, while referring to the first database;
a second determining unit configured to determine a change tendency in an appearance or an organ of the target person, based on the determined risk level, while referring to the second database; and
an image generating unit configured to generate a future-image of the target person in an appearance or an organ, based on the appearance data or organ data and the determined change tendency,
wherein the storing unit stores the risk level and the change tendency for each type of the health-related data,
wherein the first determining unit determines the risk level for each type of the health-related data,
wherein the second determining unit determines the change tendency for each type of the health-related data, and
wherein the image generating unit generates the future-image, based on the appearance data or organ data of the target person and the change tendency determined for each type of the health-related data.

2. (canceled)

3. The future-image predicting device according to claim 1, further comprises a first display unit configured to display the future-image,
wherein the display unit is capable of further displaying at least one of a first operation part capable of continuously or stepwisely changing the health-related data of the target person and a second operation part capable of continuously or stepwisely changing the future-image,
wherein, when the health-related data is changed with the first operation part, a future-image corresponding to the changed health-related data is displayed on the first display unit, and
wherein, when the future-image is changed with the second operation part, health-related data corresponding to the changed future-image is displayed on the first display unit.

4. The future-image predicting device according to claim 1, further comprising an output unit configured to output the future-image,
wherein the first determining unit calculates a composite value of a plurality of risk levels respectively determined for a plurality of types of the health-related data,
wherein the storing unit further stores a third database which indicates a relation between the composite value and a developing-risk or a severed-risk of the predetermined disease, and
wherein the output unit outputs the developing-risk or the severed-risk of the predetermined disease corresponding to the composite value the plurality of risk levels obtained from the health-related data of the target person.

5. The future-image predicting device according to claim 1, further comprising a second display unit configured to selectably display a plurality of organ images,
wherein an organ image selected on the second display unit is entered into the input unit as the organ data of the target person.

6. A program instructions installed on and executed by a computer for controlling a computer including: a storing unit configured to store a first database which indicates a relation between health-related data and a risk level of a predetermined disease, and a second data base which indicates a relation between the risk level and a change tendency in an appearance or an organ; and an input unit into which health-related data and appearance data or organ data of a target person are entered, the program comprising:
a first step for determining a risk level of the predetermined disease for the health-related data of the target person, while referring to the first database;
a second step for determining a change tendency in an appearance or an organ of the target person, based on the determined risk level, while referring to the second database; and
a third step for generating a future-image of the target person in an appearance or an organ, based on both the appearance data or organ data and the determined change tendency,
wherein the storing unit stores the risk level and the change tendency for each type of the health-related data,
wherein, in the first step, the first determining unit determines the risk level for each type of the health-related data,
wherein, in the second step, the second determining unit determines the change tendency for each type of the health-related data, and
wherein, in the third step, the image generating unit generates the future-image, based on the appearance data or organ data of the target person and the change tendency determined for each type of the health-related data.

7. (canceled)

8. The program according to claim 6, wherein the computer further includes a first display unit configured to display the future-image,
wherein the display unit is capable of further displaying at least one of a first operation part capable of continuously or stepwisely changing the health-related data of the target person and a second operation part capable of continuously or stepwisely changing the future-image,
wherein the program further comprises a fourth step for displaying, when the health-related data is changed with the first operation part, a future-image corresponding to the changed health-related data on the first display unit, and for displaying, when the future-image is changed with the second operation part, health-related data corresponding to the changed future-image on the first display unit.

9. The program according to claim 6, wherein the computer further includes an output unit configured to output the future-image,
wherein a composite value of a plurality of risk levels respectively determined for a plurality of types of the health-related data is calculated,
wherein the storing unit further stores a third database which indicates a relation between a composite value and a developing-risk or a severed-risk of the predetermined disease, and
wherein the program further comprises a fifth step for outputting, from the output unit, the developing-risk or the severed-risk of the predetermined disease corresponding to the calculated composite value.

10. The program according to claim 6, wherein the computer further includes a second display unit configured to selectably display a plurality of organ images,
wherein an organ image selected on the second display unit is entered into the input unit as the organ data of the target person.

11. A non-transitory computer readable storage medium storing a set of program instructions installed on and executed by a computer for controlling a computer including: a storing unit configured to store a first database which indicates a relation between health-related data and a risk level of a predetermined disease, and a second data base which indicates a relation between the risk level and a change tendency in an appearance or an organ; and an input unit into which health-related data and appearance data or organ data of a target person are entered, the program comprising:
a first step for determining a risk level of the predetermined disease for the health-related data of the target person, while referring to the first database;
a second step for determining a change tendency in an appearance or an organ of the target person, based on the determined risk level, while referring to the second database; and
a third step for generating a future-image of the target person in an appearance or an organ, based on both the entered appearance data or the organ data and the determined change tendency,
wherein the storing unit stores the risk level and the change tendency for each type of the health-related data,
wherein, in the first step, the first determining unit determines the risk level for each type of the health-related data,
wherein, in the second step, the second determining unit determines the change tendency for each type of the health-related data, and
wherein, in the third step, the image generating unit generates the future-image, based on the appearance data or organ data of the target person and the change tendency determined for each type of the health-related data.

12. (canceled)

13. The non-transitory computer readable storage medium according to claim 11, wherein the computer further includes a first display unit configured to display the future-image,
wherein the display unit is capable of further displaying at least one of a first operation part capable of continuously or stepwisely changing the entered health-related data of the target person and a second operation part capable of continuously or stepwisely changing the future-image,
wherein the program further comprises a fourth step for displaying, when the health-related data is changed with the first operation part, a future-image corresponding to the changed health-related data on the first display unit, and for displaying, when the future-image is changed with the second operation part, health-related data corresponding to the changed future-image on the first display unit.

14. The non-transitory computer readable storage medium according to claim 11, wherein the computer further includes an output unit configured to output the future-image,
wherein a composite value of a plurality of risk levels respectively determined for a plurality of types of the health-related data is calculated,
wherein the storing unit further stores a third database which indicates a relation between a composite value and a developing-risk or a severed-risk of the predetermined disease, and
wherein the program further comprises a fifth step for outputting, from the output unit, the developing-risk or the severed-risk of the predetermined disease corresponding to the calculated composite value.

15. The non-transitory computer readable storage medium according to claim 11, wherein the computer further includes a second display unit configured to selectably display a plurality of organ images,
wherein an organ image selected on the second display unit is entered into the input unit as the organ data of the target person.
